(19) United States
(12) Patent Application Publication
(10) Pub. No.: US 2015/0030122 A1
(43) Pub. Date: Jan. 29, 2015

(54) METHOD FOR DUAL-ENERGY MAMMOGRAPHY

(71) Applicants: Viacheslav Gorshkov, Moscow (RU); Ravil Nazirov, Moscow (RU); Viacheslav Rodin, Moscow (RU); Nadezhda Rozhkova, Moscow (RU); Sergey Prokopenko, Moscow (RU)

(72) Inventors: Viacheslav Gorshkov, Moscow (RU); Ravil Nazirov, Moscow (RU); Viacheslav Rodin, Moscow (RU); Nadezhda Rozhkova, Moscow (RU); Sergey Prokopenko, Moscow (RU)

(21) Appl. No.: 14/360,619

(22) PCT Filed: Mar. 11, 2013
(86) PCT No.: PCT/IB2013/000344

§ 371 Date: May 25, 2014

(30) Foreign Application Priority Data
Mar. 11, 2012 (RU) 2012108684
Jul. 31, 2012 (EP) 12178674.3

(51) Int. Cl. A61B 6/00

(52) U.S. Cl. CPC A61B 6/502; A61B 6/482; A61B 6/5205
USPC 378/37

(57) ABSTRACT

The invention relates to a method for dual-energy mammography. To enable earlier recognition of the microcalcifications as precursors of an oncological tumor in the breast, the invention provides that a comparison pattern with known distributions for density, thickness and effective atomic number are disposed next to the breast; that based on the comparison pattern, the parameters of the relationship between the atomic number and the difference and ratio of the logarithms of the number of photons that flow through the breast without cooperation at two different radiation energies are determined; and that based on this relationship, the distribution of the atomic numbers in the breast are visually displayed.

### CROSS-REFERENCE TO RELATED APPLICATION

This application is a national stage application and claims the benefit of the priority filing date in PCT/IB2013/000344 referenced in WIPO Publication WO/2013/136150 filed on Mar. 11, 2013. The earliest priority date claimed is Mar. 11, 2012.

### FEDERALLY SPONSORED RESEARCH

Not Applicable

### SEQUENCE LISTING OR PROGRAM

Not Applicable

### BACKGROUND

The invention relates to a method for dual-energy mammography as generically defined by the preamble to claim 1.

The invention can be used in medicine, specifically in methods for diagnosis of benign and malignant diseases of the breast.

Microcalcifications are the precursors of an oncological tumor in the breast. They have a notably greater effective number (Z=12-14), compared to the effective atomic number of healthy tissue (Z=6.5-7.5). The presence of microcalcifications is fundamentally a sufficient prerequisite for the formation of an oncological tumor. Microcalcifications with a size below 200 μm are especially dangerous, since they are not currently detectable in a breast x-ray.

A cancerous tumor also has an elevated effective atomic number. This is associated with a different distribution of carbon and oxygen (Antoniassi M., Conceição A.L.C. Study of effective atomic number of breast tissues determined using the elastic to inelastic scattering ratio//Nuclear Instruments and Methods in Physics Research Section A: Accelerators, Spectrometers, Detectors and Associated Equipment. 2011. V. 652, No. 1, pp. 739-743).

The method according to the invention for dual-energy mammography makes it possible for microcalcifications to be detected more reliably than before and at earlier stages of disease than before, and to display an oncological new development with higher resolution than before, compared to what was possible with conventional diagnostic methods.

The most conclusive diagnostic method in early forms of cancer that are not yet palpable is x-ray screening mammography. This method is based on the effect that the degree of x-ray absorption of various tissues differs. This means the display of the quantitative distribution of photons that have flowed through the breast without any cooperation whatsoever:

$$N = {N}_{0} {e}^{- {\int }_{0}^{d} μ ( E , Z , x ) ρ ( x ) d x} \qquad ( 1 )$$

in which
N₀ is the initial number of photons,
μ(E, Z, x) is the mass coefficient distribution of the total absorption coefficients over the beam line (the mass absorption coefficient is dependent on the energy of the initial photon and on the effective atomic number of the portion of the breast),
ρ(x) is the density distribution along the radiation vector, and
d is the breast thickness along the radiation vector.

The mass absorption coefficient is proportional to the effective atomic number (within its narrow range of variation).

Thus the conventional x-ray mammogram is the display of the nonlinear distribution of the product of thickness, density, and effective atomic number in the breast.

FIG. 1a shows one example of a conventional mammogram of the breast that has microcalcifications.

The variation in density of the breast (ducts, vessels, benign formations, etc.) often hides the small microcalcifications that may be present in the mammograms. Despite the collimators employed, the detectors do detect the scattered X-radiation. The breast is therefore not imaged sharply enough in the mammograms. This in turn makes small microcalcifications even more difficult to detect. Only microcalcifications larger than 200 μm can be reliably identified. Smaller microcalcifications are detectable only in homogeneous artificial specimens (phantoms).

To enhance the sensitivity of mammograms to the distribution of the effective atomic number, the method for dual-energy differential mammography (dual-energy subtraction mammography) is employed. It is protected by numerous patents (Dual energy rapid switching imaging system, U.S. Pat. No. 4,541,106, 1985; Dual-energy system for quantitative radiographic imaging, U.S. Pat. No. 5,150,394, 1992; Dual energy x-ray imaging system and method for radiography and mammography, U.S. Pat. No. 6,683,934, 2004).

The method for dual-energy differential mammography is described in particular detail in the following publication: Lewin, J. M., Isaacs, P. K., Vance, V., Larke, F. J.: Dual-energy contrast-enhanced digital subtraction mammography: Feasibility, Radiology, Volume 229, Number 1, 261-268, 2003.

By this method, two mammograms are produced using two different energies in the initial radiation. After that, they are logarithmized and subtracted (differentiation):

$$\begin{matrix}α = ln \frac{{N}_{0}^{L}}{{N}^{L}} - ln \frac{{N}_{0}^{H}}{{N}^{H}} \\ = ρ d ( {μ}_{L} - {μ}_{H} ) \\ = ρ d ( {k}_{α} Z + {a}_{α} )\end{matrix} \qquad ( 2 )$$

in which
L, H are indexes which correspond to the lower and upper energy value, and
k_{α, α}_{α} are linear coefficients.

Thus dual-energy differential mammography represents the visual display of the linear distribution of the product of the effective atomic number and the density.

FIG. 1b shows an example of a differential mammogram for the same breast.

The dual-energy differential mammogram is notably sharper, because the stray radiation is suppressed. However, the display of individual points in the breast does depend on both on the effective atomic number and the density and thickness of the breast. This likewise makes it more difficult to detect tiny microcalcifications (only larger microcalcifications are visible).

To lessen the influence of variations in density and thickness in the mammogram, a method for dual-energy dividing mammography has been proposed (Method for Differential Diagnosis of the Breast, Russian Patent 2391909, 2008).

This method for dual-energy dividing mammography is described especially extensively in the following publications:
1. V. A. Gorshkov, N. I. Rozhkova, S. P. Prokopenko. Zwei-Energien-Divisions-Mammographie. Verfahren zur nichtlinearen Analyse für Kardiologie und Onkologie. Physikalische Ansatze und klinische Praxis. Ausgabe 2. [Dual-energy dividing mammography. Method for nonlinear analysis for cardiology and oncology. Physical principles and clinical practice. 2nd edition] published by OOO KDU Verlag, 2010, pp. 173-191.
2. V. Gorshkov, N. Rozhkova, S. Prokopenko. Dual-energy-dividing-mammography, International Workshop on Digital Mammography, 2010. Girona, Spain, pp.606-613.

In dual-energy dividing mammography, the ratio of the aforementioned logarithms is displayed:

$$\begin{matrix}β = \frac{{μ}^{L}}{{μ}^{H}} \\ = \frac{ln \frac{{N}_{0}^{L}}{{N}^{L}}}{ln \frac{{N}_{0}^{H}}{{N}^{H}}} \\ = {k}_{β} Z + {a}_{β }\end{matrix} \qquad ( 3 )$$

It can be seen from this that this ratio is not dependent on the density. It is determined only by the distribution of the effective atomic numbers.

FIG. 1c shows one example of a dividing mammogram of the same breast.

The variations in density and thickness are less pronounced in the dividing mammogram than in the differential mammogram. The nipple of the breast is practically not visible in the differential mammogram (it has a very slight thickness and is therefore shown in the dividing mammogram with practically the same light density as the breast).

However, the mammogram shown in FIG. 1c shows vessels and ducts and other variations in density. This is associated with the fact that equation (3) applies only to a radiation spectrum of uniform energy. In real spectra of the x-ray tube, which contain both characteristic radiation and Bremsstrahlung, also called braking radiation, it is impossible to suppress the variations in density and thickness in the mammograms.

For effective diagnosis of breast diseases, a visual display of the effective atomic number, the density, and their convex combination is a prerequisite.

### SUMMARY

It is the object of the invention to develop a method for dual-energy mammography which makes it possible to detect smaller microcalcifications than before.

This object is attained by the features of claim 1.

To obtain mammograms with the following displayed distributions:
an effective atomic number that is invariant relative to the variation in density,
a density of the effective atomic number that is invariant relative to the variation,
the convex combination of the effective atomic number and the density,
a method according to the invention for dual-energy differential and dividing mammography is proposed.

### DRAWINGS

The invention will be described in further detail in conjunction with the drawings. In the drawings:

FIG. 1 shows examples of mammograms

FIG. 1a—x-ray screening mammography,

FIG. 1b—dual-energy differential x-ray mammography,

FIG. 1c—the same for dividing mammography,

FIG. 1d—the same for dividing-differential mammography with the distribution of the effective atomic number

FIG. 2 shows a portion of a conventional mammogram (FIG. 2a) and of a dividing-differential mammogram (distribution of the effective atomic number) (FIG. 2b)

FIG. 3 shows sections of the mammograms:

FIG. 3a—in x-ray screening mammography;

FIG. 3b—in dual-energy differential x-ray mammography;

FIG. 3c—the same for dividing mammography;

FIG. 4d—the same for divisional-differential mammography, with a distribution of the convex combination of the effective atomic number and density, and

FIG. 4 shows a diagram of the method for ascertaining the distribution of the effective atomic number, of the density, and of the convex combination thereof.

### DETAILED DESCRIPTION

From this it follows that:

In the continuous spectra, the numerical values—both for the differences and for the ratios of the aforementioned logarithms, are associated linearly with both the density (at constant thickness) and with the effective atomic number (within its narrow range of variation). Consequently, it is possible to display the effective atomic number and the density based on the following equations:

$$\begin{matrix}\begin{matrix}Z = {k}_{α} α + {k}_{β} β + {k}_{0} \\ = {k}_{a} ( ln \frac{{N}_{0}^{L}}{{N}^{L}} - ln \frac{{N}_{0}^{H}}{{N}^{H}} ) + {k}_{β} \frac{ln \frac{{N}_{0}^{L}}{{N}^{L}}}{ln \frac{{N}_{0}^{H}}{{N}^{H}}} + {k}_{0} ,\end{matrix} & ( 4 ) \\ \begin{matrix}ρ = {k}_{α}^{ρ} α + {k}_{β}^{ρ} β + {k}_{0}^{ρ} \\ = {k}_{α}^{ρ} ( ln \frac{{N}_{0}^{L}}{{N}^{L}} - ln \frac{{N}_{0}^{H}}{{N}^{H}} ) + {k}_{β}^{ρ} \frac{ln \frac{{N}_{0}^{L}}{{N}^{L}}}{ln \frac{{N}_{0}^{H}}{{N}^{H}}} + {k}_{0}^{ρ}\end{matrix} & \end{matrix}$$

The distribution of the convex combination of their uniform variables is defined as

λ=kZₙ+(1−k)ρₙ

in which k is the coefficient (0≦k≦1).

Standardization of the effective atomic number:

$${Z}_{n} = \frac{( Z - {Z}_{min} )}{( {Z}_{max} - {Z}_{min} )} ,$$

Standardization of the effective density:

$${ρ}_{n} = \frac{( ρ - {ρ}_{min} )}{( {ρ}_{max} - {ρ}_{min} )}$$

The problem exists in determining the coefficients k_{α}^{z}, k_{β}^{z}, k₀^{z}, k_{α}^{ρ}, k_{β}^{ρ}, k₀^{ρ}. To estimate these coefficients, in addition to the mammography a comparison pattern with known distributions for the density, thickness and effective atomic number is used (its characteristic values are similar to the characteristic curve for the breast). These coefficients are ascertained from the mammograms of the comparison pattern with two kinds of energy.

In WO 99/45371, a method in computed tomography is described in which a comparison pattern is positioned next to a body part that is to be examined.

Both this effect and the numerical restoration of the distribution of the effective atomic number and of the density are the definitive features of the method of the invention.

In this specification, N, β, α, Z, ρ, Zₙ, ρₙ, λ are matrixes which define the values of the corresponding characteristic variables as the i^{th}, j^{th} pixel of a detector (of the mammogram).

The coefficients k_{α}^{z}, k_{β}^{z}, k₀^{z}, k_{α}^{ρ}, k_{β}^{ρ}, k₀^{ρ} are scalar variables.

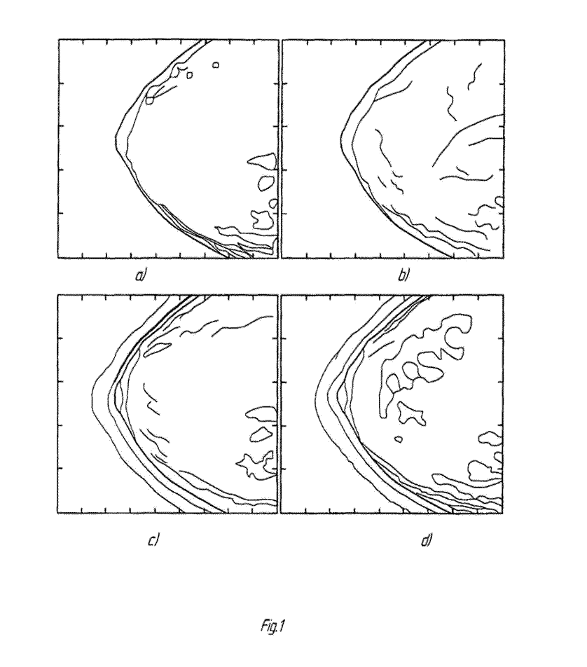

FIG. 1d shows an example for a distribution of the effective atomic number which has been ascertained on the basis of two mammograms with the aid of the dual-energy divisional-differential mammography of the invention. These two mammograms were generated at two different plate voltages of the x-ray tube. As a comparison pattern, a graphic prism (simulating tissue of the breast) with aluminum strips of various thickness (simulating microcalcifications) was employed.

In the divisional-differential mammogram, practically no vessels and ducts are visible any longer. The healthy parts of the breast are displayed with the same light density.

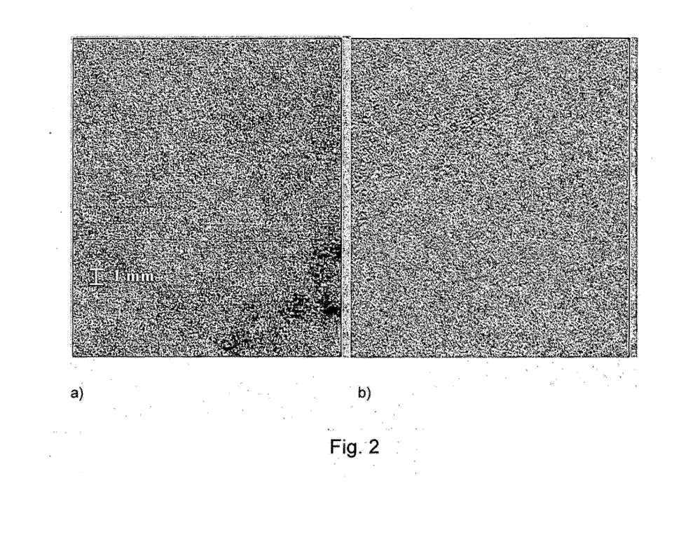

FIG. 2 shows excerpts from a conventional mammogram (FIG. 2a) and a dual-energy divisional-differential mammogram (FIG. 2b) (distribution of the effective atomic number and of the density). Large microcalcifications can be detected well enough in the conventional mammogram. However, in it the small microcalcifications, which are readily visible in the divisional-differential mammogram, are not visible. Some clusters of small microcalcifications in the divisional-differential mammogram look like merely a large granule in the conventional mammogram.

Both a cancer tumor and the microcalcifications have not only the elevated effective atomic number, but also an increased density. Therefore such inclusions can be better identified by means of a convex combination of their uniform variables (normal values).

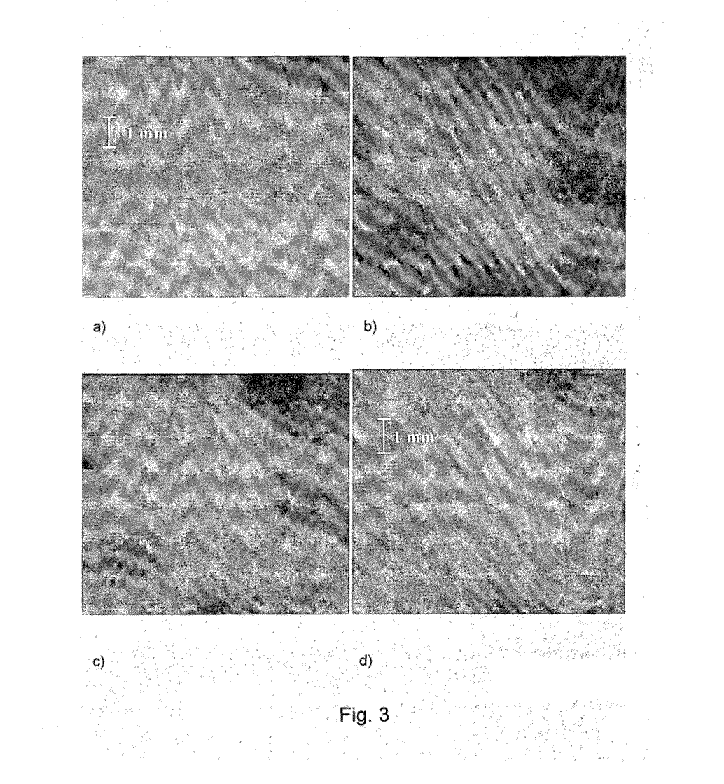

FIG. 3 describes the effectiveness of the display of the convex combination of the identified uniform variables of the effective atomic number and of the density. It can be seen from this that the tiniest microcalcifications (which are not detectable in conventional screening mammography and are detectable only with difficulty in the differential mammogram) can be identified here effectively enough in the distribution of the convex combination of the uniform variables of the effective atomic number and of the density.

Thus divisional-differential mammography makes it possible for the diseases of the breast that can be ascribed to the formation of microcalcifications to be detected in an earlier stage of their development.

The invention is performed in the following steps:
1. The comparison pattern with the known density, thickness and atomic number distributions is placed next to the breast on the plate of the mammography machine.
2. Two mammograms are made, at low and high anode voltage.
3. The coefficients from formula 4 are calculated on the basis of the comparison pattern.
4. The distribution of the effective atomic numbers, of the density, and their convex combination in the breast is displayed visually with the aid of the calculated coefficients.

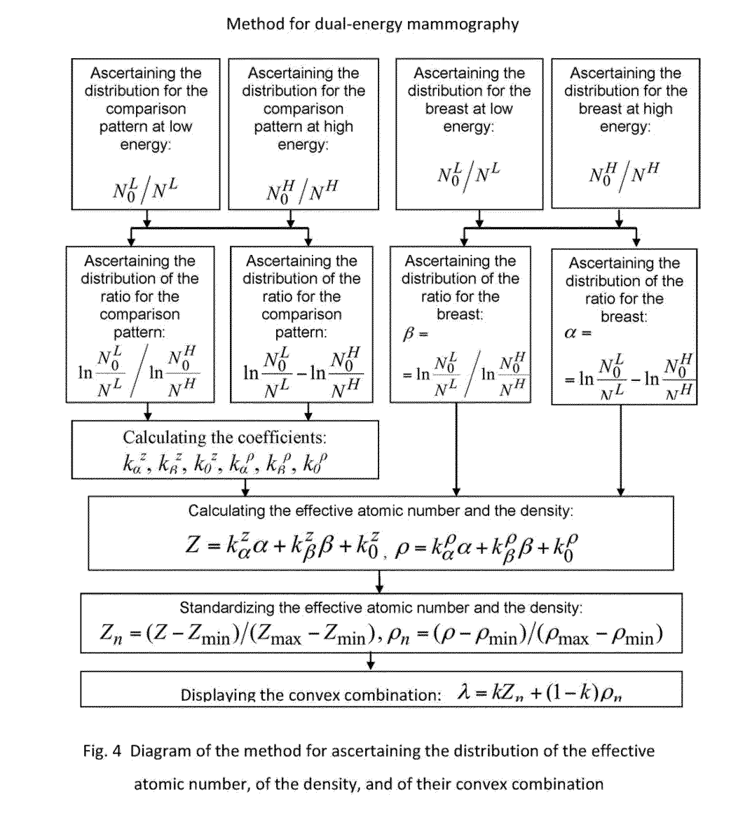

FIG. 4 shows the functional principle for performing the method for dual-energy divisional-differential mammography.
1. The comparison pattern with the known density, thickness and atomic number distributions is placed next to the breast on the plate of the mammography machine.
2. Two mammograms are made, at low and high anode voltage. With their aid, the distributions of the ratios of the initial photon quantity N₀ for the photon quantity detected by the detector as well as for the comparison pattern and for the breast are ascertained at low energy (L) and high energy (H).
3. Based on the distributions ascertained, the logarithmic distributions of the ratios and the differences are ascertained for the comparison pattern and for the breast:

$$ln \frac{{N}_{0}^{L}}{{N}^{L}} / ln \frac{{N}_{0}^{H}}{{N}^{H}} , ln \frac{{N}_{0}^{L}}{{N}^{L}} - ln \frac{{N}_{0}^{H}}{{N}^{H}}$$

4. Based on the distributions of the logarithmic ratios and differences for the comparison pattern, the coefficients k_{α}^{z}, k_{β}^{z}, k₀^{z}, k_{α}^{ρ}, k_{β}^{ρ}, k₀^{ρ} of the relationship between the effective atomic number and the density along with the ratio and these logarithms are calculated in the following equations:

$$Z = {k}_{α}^{z} ( ln \frac{{N}_{0}^{L}}{{N}^{L}} - ln \frac{{N}_{0}^{H}}{{N}^{H}} ) + {k}_{β}^{z} \frac{ln \frac{{N}_{0}^{L}}{{N}^{L}}}{ln \frac{{N}_{0}^{H}}{{N}^{H}}} + {k}_{0}^{z}$$

$$ρ = {k}_{α}^{ρ} ( ln \frac{{N}_{0}^{L}}{{N}^{L}} - ln \frac{{N}_{0}^{H}}{{N}^{H}} ) + {k}_{β}^{ρ} \frac{ln \frac{{N}_{0}^{L}}{{N}^{L}}}{ln \frac{{N}_{0}^{H}}{{N}^{H}}} + {k}_{0}^{ρ}$$

5. Based on the ascertained coefficients, the distributions of the ratios (β) and the difference (α) of the logarithms are converted into the distributions of the effective atomic number and of the density:

Z=k_{α}^{z}α+k_{β}^{z}β+k₀^{z}

ρ=k_{α}^{ρ}α+k_{β}^{ρ}β+k₀^{ρ}

These ratios are displayed for the diagnosis.
6. Performing the standardization of the effective atomic number and the standardization of the effective density:

$${Z}_{n} = \frac{( Z - {Z}_{min} )}{( {Z}_{max} - {Z}_{min} )} , \\ {ρ}_{n} = \frac{( ρ - {ρ}_{min} )}{( {ρ}_{max} - {ρ}_{min} )}$$

7. Based on the distributions of the effective atomic number and of the density, their convex combination is calculated and visually displayed:

λ=kZₙ+(1−k)ρₙ

What is claimed:

1. A method for dual-energy mammography, with the generation of a mammogram with two different radiation energies, characterized in that a comparison pattern with known distributions of density, thickness and effective atomic number, is disposed next to the breast; that on the basis of the mammograms of the comparison pattern, which have been generated at different energies, the parameters of the combination of the atomic number with the difference and with the ratio of the logarithms of the number of photons that flow through the breast without cooperating at two different radiation energies, are determined; and that based on the parameters of this combination, the distribution of the atomic number in the breast is displayed visually on the basis of the equation
$$Z = {k}_{α}^{z} α + {k}_{β}^{z} β + {k}_{0}^{z}$$
$$Z = {k}_{α}^{z} ( ln \frac{{N}_{0}^{L}}{{N}^{L}} - ln \frac{{N}_{0}^{H}}{{N}^{H}} ) + {k}_{β}^{z} \frac{ln \frac{{N}_{0}^{L}}{{N}^{L}}}{ln \frac{{N}_{0}^{H}}{{N}^{H}}} + {k}_{0}^{z}$$
and the density based on the equation
$$ρ = {k}_{α}^{ρ} α + {k}_{β}^{ρ} β + {k}_{0}^{ρ}$$
$$ρ = {k}_{α}^{ρ} ( ln \frac{{N}_{0}^{L}}{{N}^{L}} - ln \frac{{N}_{0}^{H}}{{N}^{H}} ) + {k}_{β}^{ρ} \frac{ln \frac{{N}_{0}^{L}}{{N}^{L}}}{ln \frac{{N}_{0}^{H}}{{N}^{H}}} + {k}_{0}^{ρ}$$
in which
Z is the effective atomic number and
ρ is the effective density,
α, β are correspondingly the difference α and the ratio β of the logarithms,
N₀ is the initial number of photons and
N is the detected number of photons,
L, H are indexes that designate the lower and the higher energy, respectively, and
K_{α}^{z}, k_{β}^{z}, k₀^{z}, k_{α}^{ρ}, k_{β}^{ρ}, k₀^{ρ} are linear coefficients.

2. The method of claim 1, characterized in that, based on the distributions of the effective atomic number and the effective density, the convex combination is calculated and visually displayed in accordance with the equation
λ=kZₙ+(1−k)ρₙ
in which
k is a coefficient 0<k<1 and
Zₙ is the standardized value of the effective atomic number, which is determined in accordance with
$${Z}_{n} = \frac{( Z - {Z}_{min} )}{( {Z}_{max} - {Z}_{min} )} ,$$
and
ρₙ is the standardized value of the effective density, which is determined in accordance with
$${ρ}_{n} = \frac{( ρ - {ρ}_{min} )}{( {ρ}_{max} - {ρ}_{min} )}$$
and
Zₘᵢₙ, Zₘₐₓ, ρₘᵢₙ, ρₘₐₓ correspondingly stand for minimal and maximal values of the effective atomic number and of the effective density of the breast.
